(19) United States
(12) Patent Application Publication
(10) Pub. No.: US 2025/0182863 A1
(43) Pub. Date: Jun. 5, 2025

(54) SYSTEM AND METHOD FOR DYNAMICALLY ADJUSTING ANALYTICAL PRECISION IN CLINICAL DIAGNOSTIC PROCESSES

(71) Applicant: Bio-Rad Laboratories, Inc., Hercules, CA (US)

(72) Inventors: John Yundt-Pacheco, Fairview, TX (US); Marco Flamini, Vallejo, CA (US); Aubrey Cox, El Cerrito, CA (US)

(73) Assignee: Bio-Rad Laboratories, Inc., Hercules, CA (US)

(21) Appl. No.: 18/845,143

(22) PCT Filed: Mar. 8, 2023
(86) PCT No.: PCT/US23/63897

§ 371 Date: Sep. 9, 2024

Related U.S. Application Data
(60) Provisional application No. 63/269,074, filed on Mar. 9, 2022.

(51) Int. Cl. G16H 10/40; G16H 15/00

(52) U.S. Cl. CPC G16H 10/40; G16H 15/00

(57) ABSTRACT

Disclosed is a system and method for dynamically adjusting analytical precision of a clinical diagnostic process. The system and method employ a clinical diagnostic analyzer that evaluates a sample and determines whether the resultant value, or mean value, is within a predetermined window or within a threshold of a predetermined value of a precision profile. If so, the analyzer automatically and dynamically determines a desired analytical precision and conducts additional testing of replicate samples to achieve a desired precision and reports the results to a user.

### CROSS-REFERENCE TO RELATED APPLICATIONS

This application is a 371 national phase of, and claims the benefit of, International Application Number PCT/US2023/63897, filed Mar. 8, 2023, and U.S. Provisional Patent Application No. 63/269,074, filed Mar. 9, 2022, the disclosure of which is hereby incorporated herein in its entirety by reference.

### BACKGROUND OF THE INVENTION

The present invention relates to generally to clinical diagnostic processes, and more particularly to systems and methods for dynamically adjusting analytical precision in such processes using clinical diagnostic analyzers and systems and peer groups comprising such analyzers.

Clinical diagnostic laboratories rely on their processes and laboratory instruments, such as clinical diagnostic analyzes, to provide accurate results so that doctors, medical professionals, and patients can make informed decisions with respect to patient health and patient care.

In performing clinical diagnostic tests, such as testing patient specimens, and in providing results of those tests, it is important that the test results are meaningful and useful, and that those results are both accurate and precise. Accuracy generally refers to the closeness of a test result to an actual or true value, while precision generally refers to consistency, or the extent to which repeated test results agree with one another. The analytical precision of clinical diagnostic processes, clinical diagnostic analyzers, and the like is typically evaluated in terms of the range or spread of test results, thus, the analytical precision is inherently related to the standard deviation of repeated or replicate measurements.

Typical clinical diagnostic tests and processes thus have the analytical precision that is inherent to the defined test method, i.e., the analytical precision of the test is fixed. If a different precision is required or desired, a different defined test having a different analytical precision is needed. Because each additional evaluation of a patient specimen incurs additional cost, different test methods are used in different clinical scenarios. For example, a less-expensive screening test to determine whether someone is potentially diabetic does not require the analytical precision of a more-expensive therapeutic monitoring test to determine if a patient had made incremental progress in treatment of that condition.

Similarly, the inherent analytical precision of a test method may be sufficient at certain analytical concentrations but insufficient at other analytical concentrations, particularly at concentrations related to critical medical decision limits. Thus, while useful, current clinical diagnostic processes require multiple separate test methods, each having a desired inherent analytical precision.

### BRIEF SUMMARY OF THE INVENTION

The present invention is directed to a system and method for dynamically adjusting analytical precision in clinical diagnostic processes employing clinical diagnostic analyzers and in systems, groups of systems, and peer groups of systems employing clinical diagnostic analyzers.

In an exemplary embodiment, the system and method of the present invention use one or more clinical diagnostic analyzers to conduct testing on patient specimen samples and dynamically adjust the analytical precision of the test depending on different analytical concentrations or depending on different clinical scenarios.

In one aspect, the system and method of the present invention evaluation multiple replicate samples and reports the mean of the evaluated replicates. The analytical precision is calculated by taking the SD (standard deviation) of the evaluated samples divided by the square root of the number of samples evaluated. Thus, each additional repetition of evaluations reduces the analytical imprecision by a factor of 1 n, where n is then number of evaluations completed when the mean is reported.

For example, if a sample is tested two (2) times, the analytical imprecision is decreased by a factor of 0.707 and the CV (coefficient of variation) of the mean will thus be 0.707*CV of an individual evaluation. If a sample is tested three (3) times, the analytical imprecision is decreased by a factor of 0.577 and the CV of the mean will be 0.577*CV of an individual evaluation. And, if a sample is tested four (4) times, the analytical imprecision is decreased by a factor of 0.5 and the CV of the mean will be 0.5*CV of an individual evaluation, and so forth. Thus, the analytical imprecision can be driven lower by testing sufficient replicates of the patient sample.

In one exemplary embodiment the clinical diagnostic analyzer evaluates a sample and determines whether the resultant value, or mean value, is within a predetermined window or within a threshold of a predetermined value of a precision profile. If so, the analyzer automatically and dynamically determines a desired analytical precision and conducts additional testing of replicate samples to achieve a desired precision and reports the results to a user. In further embodiments, the analyzer reports interim results of the analyses to a user. In other exemplary embodiments a user selects a desired analytical precision for a test and the clinical diagnostic analyzer dynamically adjusts the number of replicates tested to achieve the required precision.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings and claims. In the drawings, like reference numbers indicate identical or functionally similar elements.

### BRIEF DESCRIPTION OF THE DRAWINGS

The present invention will be described in greater detail in the following detailed description of the invention with reference to the accompanying drawings that form a part hereof, in which:

FIG. 1 depicts a block diagram of a clinical diagnostic analyzer system having a plurality of clinical diagnostic analyzers in communication with a server over a network in accordance with an exemplary embodiment of the present invention.

FIG. 2 depicts a block diagram of a single clinical diagnostic analyzer of the system of FIG. 1.

FIG. 3A is a depiction of a first exemplary prompt screen presented by the clinical diagnostic analyzer of FIG. 2.

FIG. 3B is a depiction of a second exemplary prompt screen presented by the clinical diagnostic analyzer of FIG. 2.

FIG. 3C is a depiction of a third exemplary prompt screen presented by the clinical diagnostic analyzer of FIG. 2.

FIG. 4 is a block diagram of a plurality of clinical diagnostic analyzers as in FIG. 1 arranged in a peer group configuration.

FIG. 5 is a flow diagram of an exemplary method for creating a precision profile for use in a clinical diagnostic process in accordance with an exemplary embodiment of the present invention.

FIG. 6 is a flow diagram of an exemplary method for dynamically adjusting an analytical precision to meet clinical requirements in accordance with an exemplary embodiment of the present invention.

### DETAILED DESCRIPTION OF EXEMPLARY EMBODIMENTS

Systems and methods for dynamically adjusting analytical precision in clinical diagnostic processes in a clinical diagnostic analyzer, in groups of clinical diagnostic analyzers, in systems comprising clinical diagnostic analyzers, and in systems of clinical diagnostic analyzers connected in peer groups in accordance with exemplary embodiments of the present invention are described herein. While the invention will be described in detail hereinbelow with reference to the depicted exemplary embodiments and alternative embodiments, it should be understood that the invention is not limited to the specific configurations shown and described in these embodiments. Rather, one skilled in the art will appreciate that a variety of configurations may be implemented in accordance with the present invention.

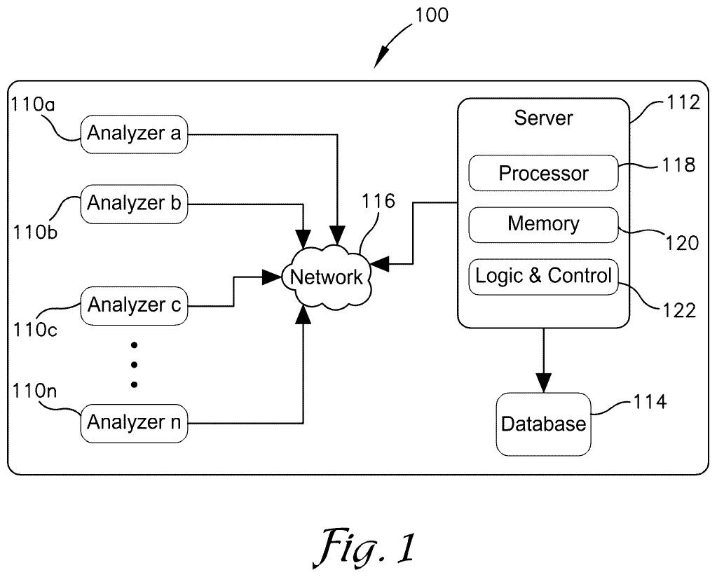

Looking first to FIG. 1, a clinical diagnostic system in accordance with an exemplary embodiment of the present invention is depicted generally by the numeral 100. The system 100 generally includes a plurality of clinical diagnostic analyzers 110a, 110b, 110c, 110n and a server 112 in communication with a database 114. The plurality of clinical diagnostic analyzers 110a, 110b, 110c, 110n are in communication with network 116, which facilitates the transmission of instructions, information, and data between each clinical diagnostic analyzer 110a, 110b, 110c, 110n and the server 112, as well as between each of the clinical diagnostic analyzers 110a, 110b, 110c, 110n and any of the other diagnostic analyzers, or between any combination of clinical diagnostic analyzers and/or the server.

Network 116 may be any local area network (LAN), wide area network (WAN), ad-hoc network, or other network configuration known in the art, or combinations thereof. For example, in the exemplary embodiment depicted in FIG. 1, network 116 may include a LAN allowing communication between the clinical diagnostic analyzers 110a, 110b, 110c, 110n, such as in a single laboratory setting having multiple clinical diagnostic analyzers, an may also include a WAN, such as the Internet or other wide area network, allowing communication between the LAN and the server 112 and/or between the clinical diagnostic analyzers and the server.

It should be understood that the configurations depicted in FIG. 1 is exemplary, and not limiting, and that the invention as described herein may be embodied in a single clinical diagnostic analyzer, in a group of clinical diagnostic analyzers co-located in a single laboratory or facility, and in group of clinical diagnostic analyzers that are geographically dispersed.

For example, multiple systems 100, each comprising one or more clinical diagnostic analyzers and servers may be located in a single laboratory, or in multiple laboratories dispersed across a facility or across the globe, all in communication via a WAN. It should be further understood that the present invention may be embodied in a single clinical diagnostic analyzer, or in a group of clinical diagnostic analyzers in communication with each other over a LAN or WAN, without a server or servers. These and other variations and embodiments will be apparent to those skilled in the art.

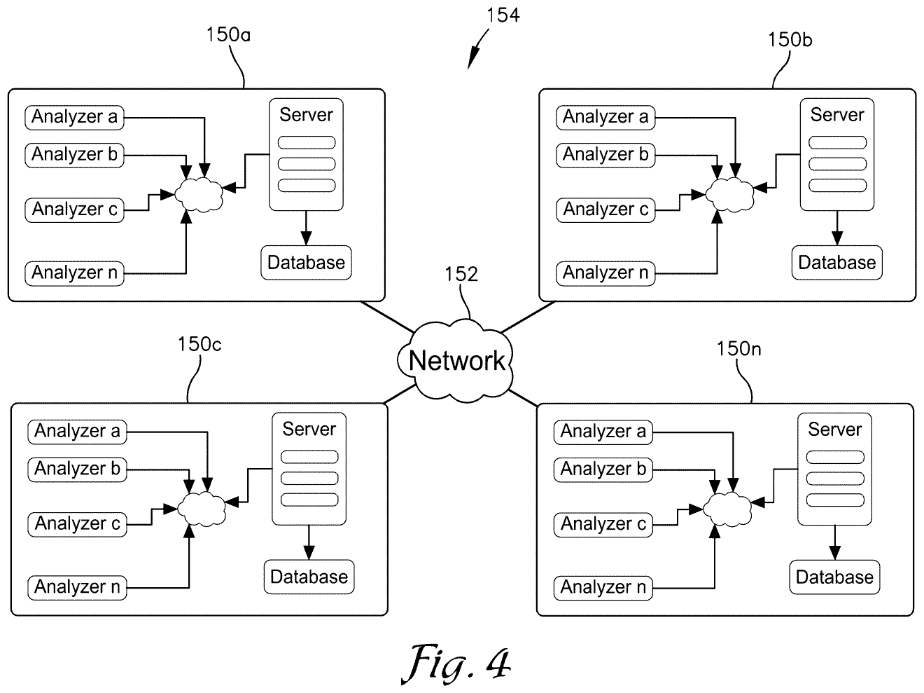

In one exemplary embodiment, as depicted in FIG. 4, a plurality of clinical diagnostics systems 150a, 150b, 150c, 150n, such as those depicted in FIG. 1, are in communication via a network, such as the Internet or other WAN. This collection of separate systems comprises a peer group of systems 152, wherein each system 150a, 150b, 150c, 150n represents a laboratory having one or more clinical diagnostic analyzers, and wherein each of the laboratories conducts testing of patient specimens and quality control materials. Most preferably, each member 150a, 150b, 150c, 150n of the peer group 152 is a laboratory at a location geographically dispersed from the other peer group member laboratories, with each laboratory having similar types of clinical diagnostic analyzers, running similar types of tests and using quality control materials similar to those used by other peer members of the peer group.

Looking back to FIG. 1, server 112 preferably includes a processor 118, memory 120, and logic and control circuitry 122, all in communication with each other. Server 112 may be any server, server system, computer, or computer system known in the art, preferably configured to communicate instructions and data between the server 112 and the network, and/or to any device connected to the network, and to store and retrieve data and information to and from the database 114. Processor 118 may be any microprocessor, controller, or plurality of such devices known in the art. Processor 118 preferably runs a server operating system such as a Linux based, Windows based, or other server operating system known in the art. Preferably, the processor 118 is configured to control the operation of the server 112 in conjunction with the operating system, allowing the server to communicate with the database 114 and the network 116 and/or with devices connected to the network, such as the clinical diagnostic analyzers 110a, 110b, 110c, 110n. In some embodiments the server may control the operation of the clinical diagnostic analyzers, for example allowing operation of the analyzers during specific time periods, collecting data from the analyzers for storage in the database 114, transferring data to the analyzers for viewing and/or analysis, collecting test data from the analyzers, and providing data, instructions or prompts to the analyzers either individually or in groups.

Memory 120 may be volatile or non-volatile memory and is used to store data and information associated with the operation of the server as well as data for transmission to and from the server. For example, the memory stores the server operating system for execution by the processor 118 and may also store data associated with the clinical diagnostic analyzers 110a, 110b, 110c, 110n in communication with the server 112 over the network 116. In some embodiments the memory 120 on the server may be used as a supplement to, or in place of, the database 114.

The database 114 is preferably used to store control information relating to the operation of the server 112 and the operation and control of the clinical diagnostic analyzers 110a, 110b, 110c, 110n, and may also be used to store data relating to the processing of samples by the clinical diagnostic analyzers. For example, the database may contain instructions or programming for execution by a processor on a clinical diagnostic analyzer, or for execution on the server, or may store data related to the number of samples processed, the frequency of testing, the results of analysis performed on the analyzer, as well as data relating to the samples themselves, such as tracking information, lot numbers, sample size, sample weight, percentage of sample remaining, and the like. Preferably, the database 114 includes non-volatile storage such as hard drives, solid state memory, and combinations thereof.

Logic and control circuitry 122 provides interface circuitry to allow the processor and memory to communicate, and to provide other operational functionality to the server, such as facilitating data communications to and from the network 116.

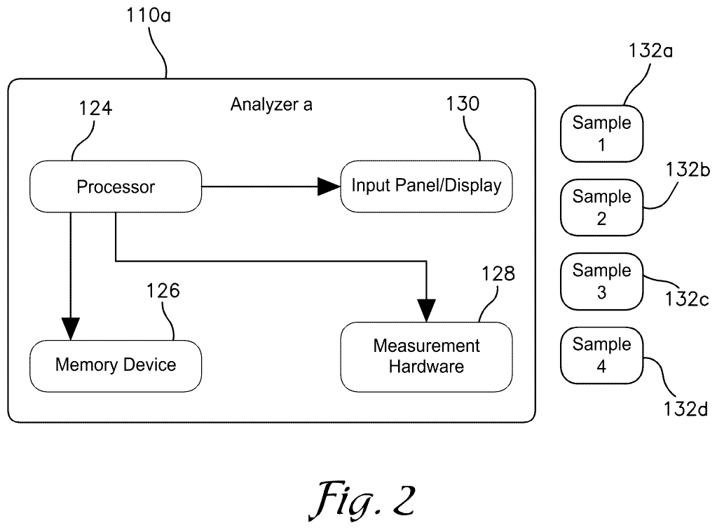

Turning to FIG. 2, a detailed view of a single clinical diagnostic analyzer 110a of the system of FIG. 1 is depicted. Clinical diagnostic analyzer 110a preferably comprises a processor 124, a memory device 126, measurement hardware 128, and an input panel/display 130.

The processor 124 may be any controller, microcontroller, or microprocessor as known in the art, and is in communication with memory device 126 which stores instructions for execution by the processor to control and communicate with the measurement hardware 128 and the input panel/display 130 to cause the clinical diagnostic analyzer to perform desired steps, such as sampling as commanding the measurement hardware to load test specimens or to perform a test on a loaded sample, or instructing or prompting an user to perform specific operations such as replacing a test sample, beginning a test, or viewing collected data. The processor 124 may also execute instructions to receive data from the measurement hardware 128 and to perform one or more analyses on the received data, and to display test results or other information on the input panel/display panel 130.

Measurement hardware 128 preferably includes a sample receptacle configured to receive one or more samples or specimens into the analyzer for testing. Preferably, the measurement hardware is configured to receive samples stored within vials, and most preferably is configured to receive a plurality of vials and to extract analytes, from any desired vial for and analysis. In further embodiments, the measurement hardware 128 may include external turntables, loaders, or other mechanisms to facilitate the loading and unloading of samples to allow samples to be loaded under command of the analyzer.

As depicted in FIG. 2, the measurement hardware is configured to be used with material samples 132a, 132b, 132c, 132d, which may be QC materials, patient test specimens or other specimens as is known in the art. In one embodiment, the material samples are contained in vials which are loaded or inserted into the clinical diagnostic analyzer 110a by a user. The samples may be loaded individually, or in groups, e.g., in a tray that is loaded into the analyzer. In alternative embodiments, the samples may be loaded using an automated loading mechanism, such as a turntable or other mechanism, upon command from the analyzer 110a. Material samples in the form of QC materials are typically provided in lots, with a unique lot number assigned to a lot of samples that are essentially identical as coming from the exact same batch source of material. The analyzer 110a preferably allows information relating to the QC materials to be entered by a user, including statistical information such as a mean or standard deviation for the lot of material. In other embodiments, the information may be obtained over a network or from a server using, for example a QR code on the sample vial or container to uniquely identify the sample or lot.

Input panel/display 130 is in communication with the processor and is operable to present controls to facilitate operation of the analyzer, as well as to present prompts and instructions to an user, and to receive input commands and/or data from the user. The input panel/display 130 is preferably a touch screen having capabilities of displaying text and graphics as well as icons, push buttons, keyboards, and the like to both present data to a user and to receive input from a user of the analyzer. Preferably, the input panel/display 130 includes an audible alert device such as a buzzer or beeper.

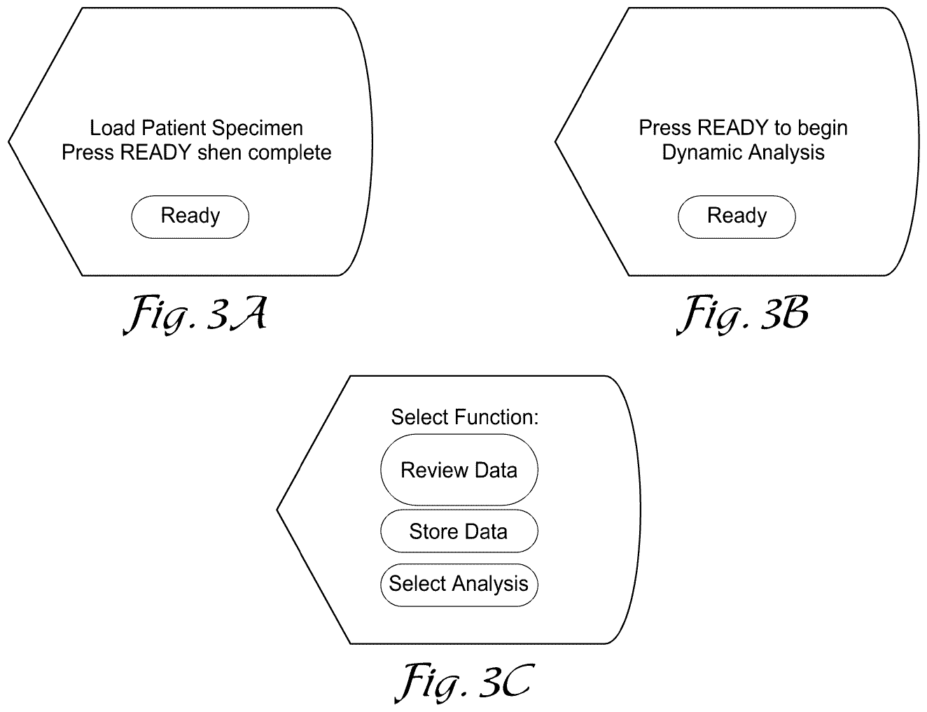

Looking to FIGS. 3A, 3B, 3C, and 3D, for example, the input panel/display may present prompts to a user to load one patient specimen and press a READY button once completed (FIG. 3A), to begin a dynamic analyses of a loaded patient specimen (FIG. 3B), or to review, select, store data, or select another analysis (FIG. 3C). It should be understood that the clinical diagnostic analyzer 100a may have multiple programs and functions available, a menu or selection prompt is preferably presented to guide a user through the operation of the analyzer and the selection of desired functions and operations.

Clinical diagnostic analyzer 110a may be any type of analyzer known in the art, such as biochemistry analyzers, hematology analyzers, immune-based analyzers, or any other clinical diagnostic analyzer known in the art. Preferably, analyzer 110a is configured to evaluate or test patient specimens or samples and to automatically and dynamically adjust or control the running of subsequent replicate evaluations to achieve a desired analytical precision of the testing. Clinical diagnostic analyzer 110a may be configured for use with various patient specimens or samples, whether in liquid or lyophilized form, and may be configured for use in the immunoassay, serum chemistry, immunology, hematology, and other fields.

Looking to FIGS. 1 through 3 in combination, in typical use in performing a test on a patient specimen, the analyzer 110a prompts a user to load a patient specimen as depicted in FIG. 3A, and to perform a dynamic analysis as depicted in FIG. 3B once the sample is loaded. Upon completion of the test, the analyzer may prompt the user to store or review the data.

It should be understood that the operation of the analyzer 100a may be performed locally, at the analyzer, or that the operation may be coordinated thorough the server 112 when the analyzer is operated in a system 100 as depicted in FIG. 1 It should be further understood that any data may be stored locally on the analyzer 110a, on the server 112 or database 114, and that the data may be made available throughout the system 100 and over the network 115 so that remote servers and analyzers may likewise access the stored data. Similarly, analyses may be run on the analyzer itself, on the server, or may be distributed among multiple analyzers and/or servers.

It should also be understood that data collected and/or stored on any of the individual clinical diagnostic analyzers in any of the systems may be shared and communicated to other clinical diagnostic analyzers in that same system or laboratory, may be shared and communicated with the server and database within that system, and may be shared and communicated to other systems, and to the clinical diagnostic analyzers and servers and databases within those other systems.

Looking to FIG. 4, in one exemplary embodiment, a plurality of clinical diagnostics systems 150a, 150b, 150c, 150n, each of which are similar to that depicted in FIG. 1, are in communication via a network 152, such as the Internet or other WAN. This collection of separate systems comprises a peer group 154 of systems, wherein each system 150a, 150b, 150c, 150n represents a laboratory having one or more clinical diagnostic analyzers, and wherein each of laboratories conducts testing of patient specimens and quality control materials. Most preferably, each member 150a, 150b, 150c, 150n of the peer group 154 is a laboratory at a location geographically dispersed from the other peer group member laboratories, with each laboratory having similar types of clinical diagnostic analyzers, running similar types of tests and using quality control materials similar to those used by other members of the peer group.

In embodiments of the invention described herein, the analyses performed on multiple analyzers and the data collected by members of the peer group may be analyzed in combination to provide an output or result based on data collected across multiple analyzers, and based on data collected by other members of the peer group.

With the configuration of clinical diagnostic analyzers, systems employing clinical diagnostic analyzers, and peer groups of clinical diagnostic analyzers set forth, systems and methods for dynamically adjusting analytical precision in clinical diagnostic processes will now be described.

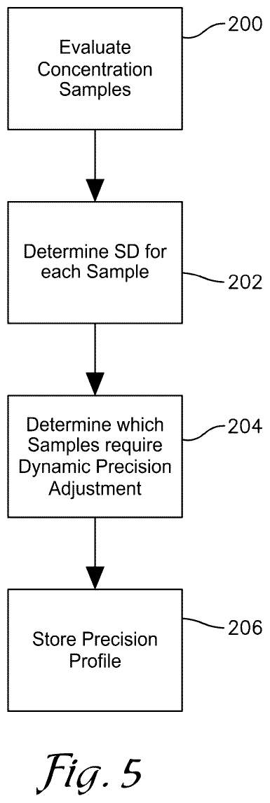

Looking first to FIG. 5, an exemplary method of creating a precision profile for a desired test method is depicted. In general, the precision profile is created by performing a precision study using samples of at least five (5) concentrations of an appropriate analyte for the desired test. Preferably the five samples comprise one sample at each end of the desired analytical range, one sample near a medical decision point (i.e., a point where a patient sample value would trigger a medical decision if the patient sample were above or below that point), and one sample on either side of the medical decision point. In alternative embodiments additional samples having other concentrations may also be included in creating the precision profile.

At block 200, samples of varying concentrations for the desired test method are evaluated. Preferably, approximately at least sixty to one-hundred and twenty replicates of each sample are evaluated, although more or fewer evaluations may be made depending on the desired degree of confidence.

At block 202, a standard deviation (SD) for each sample is determined by performing a non-linear regression analysis of the sample's SD across the analytical range, with a SD assigned to each concentration. It should be understood that this step may be performed in conjunction with the evaluation step of block 200 and that that the SD may be determined on a running basis as the evaluations progress rather than being determined only after all evaluations have been completed.

At block 204, with the SD determined and assigned for each concentration, the profile is evaluated to determine which analytical concentration ranges may benefit from better precision, and how much additional precision is required to adequately support clinical decisions. It should be understood that the evaluation may determine that no additional precision is required for a particular range, or that one or more ranges may require that one or more replicates will need to be tested and averaged with previous results to decrease the imprecision for that range to meet the desired clinical requirements.

At block 206, the precision profile for the test method is completed and stored in the clinical diagnostic analyzer, and/or stored in other networked systems or devices as previously described, for use in testing patient specimens.

For example, in creating a precision profile for a hemoglobin A1c test, it may be determined that the following adjustments should be made for various ranges:
A1c level of 5.7 to 6.4—reduce imprecision by a factor of 0.707
A1c level of 6.5 to 7.0—reduce imprecision by a factor of 0.577
A1c level 7.0 to 8.0—reduce imprecision by a factor of 0.707

Those determined factors are stored in the precision profile associated with the A1c test method. Thus, as a clinical diagnostic analyzer evaluates A1c patient samples, any results falling within those ranges will cause the analyzer to dynamically adjust the analytical precision according to the associated factors and thus to evaluate an additional number of replicates of the patient sample to achieve the desired precision, i.e., to reduce the imprecision for that range. As described previously, reducing the imprecision by a factor of 0.707 requires evaluating two replicates, and reducing the imprecision by a factor of 0.577 requires evaluating three replicates, and so forth. It should be understood that the factors, the number of replicates to run, and other corresponding data may be stored in the precision profile and retrieved by a clinical diagnostic analyzer for use.

Preferably precision profiles for multiple test methods are created and stored on the clinical diagnostic analyzer or on the laboratory network as previously described for ready access by a clinical diagnostic analyzer to be used for a particular test.

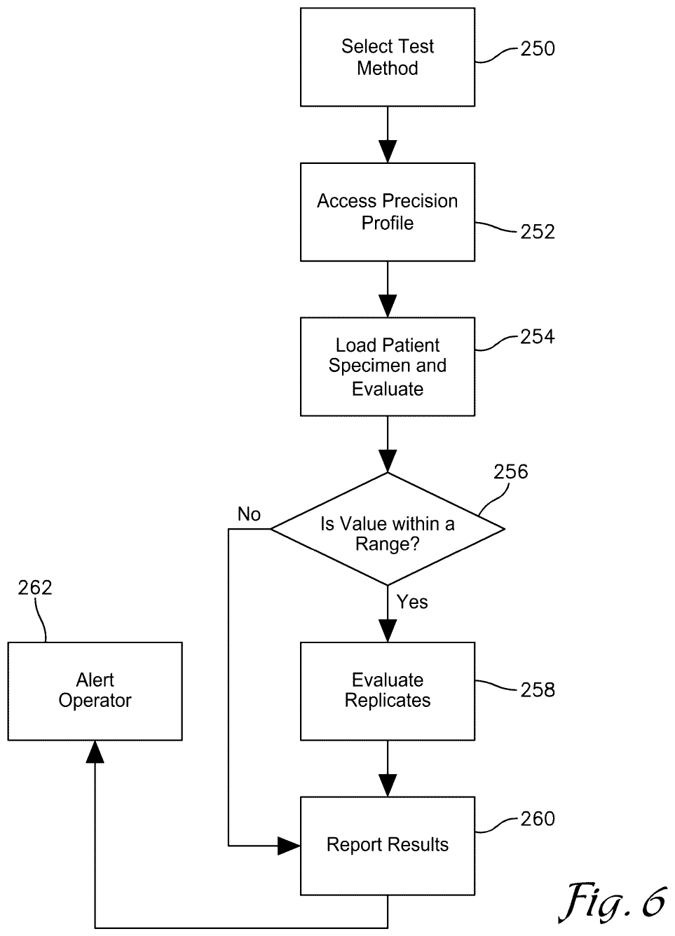

With the operation of the clinical diagnostic process and clinical diagnostic analyzer set forth, and the creation of the precision profile for a particular test method as just described, the operation of the system and method of the present invention in dynamically adjusting analytical precision will now be described with reference to FIG. 6.

Looking to FIG. 6, at block 250, a user selects a desired test method or analysis to be performed, such as by using the selection screen of a clinical diagnostic analyzer as shown in FIG. 3C. For example, a user may select to run an A1c test, a HbA2 test, or other desired test, test method, or analysis.

At block 252, with the test method selected, a precision profile associated with the selected test method is accessed by the clinical diagnostic analyzer, and the adjustment factors (or number of replicates to run) from the precision profile for the various ranges associated with the selected test method are loaded into the clinical diagnostic analyzer.

With the test method selected and an associated precision profile loaded, at block 254 a patient specimen to be tested is loaded and an analyte from that specimen is evaluated.

At block 256, the value from the evaluation is compared to the range of values in the precision profile to determine if the value is within a range identified in the precision profile as requiring additional precision, i.e., needing additional evaluations performed.

If the value at block 256 is within an identified range, at block 258, one or more additional replicates of the patient specimen are evaluated (depending on the additional precision required and called for in the precision profile) and a mean of the multiple evaluations is calculated as the result value of the evaluation. Once the evaluation of the additional replicates is completed, the process proceeds to block 260 where the results are displayed to the user and stored in the laboratory system.

If, at block 256, no additional precision is required, then no additional replicates are evaluated and the process proceeds to block 260 where the results are displayed to the user.

At block 262, if the results are outside of a predetermined window or threshold associated with selected test method, then a user alert is generated by the clinical diagnostic analyzer.

The method above thus provides dynamically adjusted analytical precision for clinical diagnostic process that is performed automatically by a clinical diagnostic analyzer without user invention or intermediate evaluation of results.

In a further example, the system and method of the present invention will be described in an exemplary use with a hemoglobin A2 (HbA2) test. High levels of hemoglobin A2 are used as a diagnostic test for Beta thalassemia, a blood disorder that reduces the production of hemoglobin. If a patient is identified as having abnormally high HbA2, that patient is typically referred for definitive molecular testing. Because molecular testing is expensive, false positives for high HbA2 are undesirable.

Using the system and method of dynamically adjusting analytical precision of the present invention, false positives for high HbA2 can be minimized by increasing the precision for samples that fall in the border zone between normal and high levels of HbA2. Thus, when a patient sample has an initial evaluation that falls within that border zone, the system and method as described above can instruct the clinical diagnostic analyzer to automatically evaluate one or more replicates to achieve the desired precision for that border zone sample. The additional evaluation is performed dynamically and automatically without any user intervention, with the higher precision result reported.

Continuing the HbA2 example, assume the precision profile for the HbA2 test method has the following values:

| Mean | CV |
|---|---|
| 2.11 | 1.98 |
| 2.33 | 2.08 |
| 2.54 | 2.24 |
| 2.64 | 2.38 |
| 4.87 | 1.03 |
| 5.16 | 1.70 |

and that the HbA2 border zone is in the range of 3.1 to 3.8.

Any samples with an initial evaluation between 3.1 and 3.8 (i.e., in the border zone) will be evaluated a second time and the mean of the two evaluations will be reported as the result. Any samples with an initial evaluation less than 3.1 or greater than 3.8 will not be evaluated again and the initial evaluation value will be reported. For example, the following samples and initial evaluations would be evaluated and reported as follows:

| Sample | Initial | Repeat | Mean | |
|---|---|---|---|---|
| ID | Value | Value | Value | Disposition |
| A | 3.1 | 2.8 | 2.95 | Normal |
| B | 3.0 | n/a | n/a | Normal |
| C | 3.2 | 2.9 | 3.05 | Normal |
| D | 3.1 | 2.9 | 3.00 | Normal |
| E | 3.3 | 3.3 | 3.30 | High |
| F | 3.7 | 3.4 | 3.55 | High |
| G | 3.4 | 3.6 | 3.50 | High |

As can be seen, the samples with an initial value within the border zone range of 3.1 to 3.8 (i.e., samples A, C, D, E, F, and G) are dynamically and automatically evaluated a second time to reduce the imprecision by a factor of 0.707, while any samples that do not have an initial value within the border zone range are not dynamically and automatically evaluated a second time. Thus, the system and method of the present invention prevents unnecessary and expensive evaluations by automatically identifying only those samples in need of additional evaluation.

As can be seen, the systems and methods of the present invention provide an improvement over known systems and methods which cannot dynamically adjust analytical precision in the course of testing patient samples.

While the present invention has been described and illustrated hereinabove with reference to various exemplary embodiments, it should be understood that various modifications could be made to these embodiments without departing from the scope of the invention. Therefore, the invention is not to be limited to the exemplary embodiments described and illustrated hereinabove, except insofar as such limitations are included in the following claims.

What is claimed and desired to be secured by Letters Patent is as follows:

1. A clinical diagnostic analyzer for dynamically adjusting analytical precision in a clinical diagnostic test, comprising:
a processor;
measurement hardware in communication with the processor and configured to measure properties of an analyte;
a memory device having stored thereon executable instructions that, when executed by the processor, cause the clinical diagnostic analyzer to perform operations comprising:
loading and evaluating a patient specimen to ascertain a first value for the specimen;
comparing the first value to determine if the first value is within a first predetermined range;
upon determining the first value is within the predetermined range, revaluating the patient specimen a predetermined number of times to determine a second value and reporting the second value to a user; and
upon determining the first value is not within the first predetermined range, reporting the first value to a user.

2. The clinical diagnostic analyzer of claim 1, wherein the first predetermined range is loaded from a precision profile data file associated with the clinical diagnostic test.

3. The clinical diagnostic analyzer of claim 2, wherein the precision profile data file comprises data comprising a first predetermined range, a second predetermined range, a predetermined number of times, a precision factor, and combinations thereof.

4. The clinical diagnostic analyzer of claim 1, wherein the executable instructions, when executed by the processor, further cause the clinical diagnostic analyzer to perform operations comprising:
generating an alert to a user when the reported first value or the reported second value are within a second predetermined range.

5. The clinical diagnostic analyzer of claim 1, wherein determining the second value comprises calculating a mean of the evaluated values.

6. A clinical diagnostic analyzer for dynamically adjusting analytical precision in a clinical diagnostic test, comprising:
a processor;
measurement hardware in communication with the processor and configured to measure properties of an analyte;
a memory device having stored thereon executable instructions that, when executed by the processor, cause the clinical diagnostic analyzer to perform operations comprising:
loading and evaluating a patient specimen to ascertain a first value for the specimen;
comparing the first value to determine if the first value is within a first predetermined range;
upon determining the first value is within the predetermined range, revaluating the patient specimen one or more times to determine a second value for the specimen;
calculating a precision value based on the first value and the second value;
reporting the precision value to a user.

7. The clinical diagnostic analyzer of claim 5, wherein the predetermined range is loaded from a precision profile data file associated with the clinical diagnostic test.

8. The clinical diagnostic analyzer of claim 6, wherein the precision profile data file comprises data comprising a predetermined range, a precision factor, and combinations thereof.

9. The clinical diagnostic analyzer of claim 1, wherein the executable instructions, when executed by the processor, further cause the clinical diagnostic analyzer to perform operations comprising:
generating an alert to a user when the reported precision value exceeds a predetermined threshold.

10. A system for dynamically adjusting analytical precision in a clinical diagnostic process, comprising:
a plurality of peer group systems, wherein each peer group system comprises:
a server comprising a processor, a memory and a database, wherein the server is in communication with servers of other peer group systems; and
a plurality of clinical diagnostic analyzers in communication with the server, wherein each of the plurality of clinical diagnostic analyzers comprises:
a processor;
measurement hardware in communication with the processor and configured to measure properties of an analyte;
a memory device having stored thereon executable instructions;
wherein the executable instructions stored on the memory device of at least one of the clinical diagnostic analyzer, when executed by the corresponding processor, cause the clinical diagnostic analyzer to perform operations comprising:
loading and evaluating a patient specimen to ascertain a first value for the specimen;
comparing the first value to determine if the first value is within a first predetermined range;
upon determining the first value is within the predetermined range, revaluating the patient specimen a predetermined number of times to determine a second value and reporting the second value to a user; and
upon determining the first value is not within the first predetermined range, reporting the first value to a user.

11. The system of claim 10, wherein the first predetermined range is loaded from a precision profile data file associated with the clinical diagnostic test.

12. The system of claim 11, wherein the precision profile data file comprises data comprising a first predetermined range, a second predetermined range, a predetermined number of times, a precision factor, and combinations thereof.

13. The system of claim 10, wherein the executable instructions, when executed by the processor, further cause the clinical diagnostic analyzer to perform operations comprising:
generating an alert to a user when the reported first value or the reported second value are within a second predetermined range.

14. The system of claim 10, wherein determining the second value comprises calculating a mean of the evaluated values.
